(19) United States
(12) Patent Application Publication
(10) Pub. No.: US 2026/0090729 A1
(43) Pub. Date: Apr. 2, 2026

(54) BLOOD PRESSURE MEASUREMENT DEVICE WITH KOROTKOFF SOUNDS RECOGNITION AND PLAYBACK FUNCTION, AND METHOD THEREOF

(71) Applicant: Beijing Hanvon Health Technology Co., LTD, Beijing (CN)

(72) Inventors: Jianmin YU, Beijing (CN); Fuzhou WANG, Beijing (CN); Rongxin HU, Beijing (CN); Yingying QU, Beijing (CN); Gang LIU, Beijing (CN); Chao WANG, Beijing (CN); Xiaoyu JIAO, Beijing (CN); Hao WANG, Beijing (CN); Longtao HAN, Beijing (CN)

(21) Appl. No.: 19/210,344

(22) Filed: May 16, 2025

(30) Foreign Application Priority Data
Sep. 30, 2024 (CN) 202411391161.8
Dec. 19, 2024 (CN) 202411886084.3

(51) Int. Cl. A61B 5/022; A61B 5/00; G16H 40/63

(52) U.S. Cl. CPC A61B 5/02208; A61B 5/7225; A61B 5/7264; A61B 5/7405; A61B 5/742; A61B 5/7475; G16H 40/63; A61B 2560/0462; A61B 2562/0204; A61B 2562/0247

(57) ABSTRACT

The present disclosure discloses a blood pressure measurement device with a Korotkoff sounds recognition and playback function, and a method thereof. The blood pressure measurement device includes an air pressure sensor, a vibration sensor and a control module; the air pressure sensor being configured to collect pressure analog signals; the vibration sensor being configured to collect brachial artery pulse signals; and the control module being configured to control the air pressure inside the air bladder and recognize Korotkoff sounds in the brachial artery pulse signals, and to obtain systolic pressure data and diastolic pressure data according to the Korotkoff sounds and the air pressure; the control module being further configured to respond to a playback operation to play back an air bladder pressure deflation process and a process from the appearance to the disappearance of the Korotkoff sounds, and display blood pressure values

### CROSS-REFERENCE TO RELATED APPLICATIONS

The present disclosure claims the priority of Chinese Patent Application No. 202411391161.8, filed on Sep. 30, 2024, and the priority of Chinese Patent Application No. 202411886084.3, filed on Dec. 19, 2024, both of which are hereby incorporated by reference in their entireties.

### TECHNICAL FIELD

The present disclosure relates to the technical field of blood pressure measurement, in particular to a blood pressure measurement device, method and electronic device, in particular a sphygmomanometer with Korotkoff sounds recognition and playback function.

### BACKGROUND

Blood pressure may provide basis for diagnosis of some diseases (such as kidney disease, endocrine disease or heart disease, etc.). A process for performing blood pressure measurement according to a Korotkoff-sound method is generally as follows: placing a stethoscope at a lower side of a cuff's air bladder and pressed against the skin, pressurizing the air bladder, blood flow in an arterial vessel of an upper arm may be blocked after air pressure in the air bladder reaches certain pressure values, then releasing gases in the air bladder to slowly lower pressure, determining a pressure value corresponding to a first Korotkoff sound as a high pressure value, and determining a pressure value corresponding to the last Korotkoff sound as a low pressure value.

In the above blood pressure measurement process, identification of Korotkoff sounds depends on hearing sense and subjective judgment of a medical staff, and is also susceptible to an impact of environmental noise, which may lead to a deviation in a final blood pressure value determined by the medical staff, affecting the accuracy of blood pressure measurement.

### SUMMARY OF INVENTION

In view of this, the present disclosure provides a blood pressure measurement device with Korotkoff sounds recognition and playback function, and a method thereof. Blood pressure values are accurately recognized by Korotkoff sounds and are effectively verified by playback of the Korotkoff sounds, so as to improve the low accuracy problem of blood pressure measurement.

In a first aspect, the present disclosure provides a blood pressure measurement device with Korotkoff sounds recognition and playback function, the blood pressure measurement device including a control module, air pressure sensor and a vibration sensor; the air pressure sensor being configured to collect pressure analog signals which are used to characterize air pressure inside an air bladder; the vibration sensor being configured to be fixed in a cuff and used to collect brachial artery pulse signals; and the control module being configured to control the air pressure inside the air bladder and recognize Korotkoff sounds in the brachial artery pulse signals collected by the vibration sensor, and obtain systolic pressure data and diastolic pressure data according to the Korotkoff sounds and the air pressure inside the air bladder; and the control module being further configured to respond to a playback operation to play back and display air pressure deflation process inside the air bladder and a process from the appearance to the disappearance of the Korotkoff sounds, and the systolic pressure data and the diastolic pressure data.

The vibration sensor is a piezoelectric sensor or a microphone.

Playback of the air pressure deflation process inside the air bladder is displayed digitally, and playback of the process from the appearance to the disappearance of the Korotkoff sounds is performed via a sound player and/or via pulsation of a heartbeat graphic symbol on a display screen.

The control module is further used to transmit the Korotkoff sounds, the air pressure inside the air bladder, the systolic pressure data and the diastolic pressure data, and a corresponding measurement time as associated blood pressure measurement data to an external device for storage and playback. At the time of a playback operation, blood pressure measurement data needing to be played back may be selected to perform the playback operation.

The blood pressure measurement device provided by this embodiment stores the Korotkoff sounds recognized during blood pressure measurement, the air pressure during air bladder pressure deflation process, the systolic pressures and the diastolic pressures obtained according to Korotkoff sounds recognition, and the measurement time, and in response to the playback operation, synchronously plays back the recognized Korotkoff sounds and the air pressure, and displays the systolic pressure and the diastolic pressure, provides a user with original measurement data of blood pressure measurement, and verifies whether a blood pressure measurement result is accurate by listening to or observing the playbacked Korotkoff sounds, and improves the accuracy of the blood pressure measurement result.

In a second aspect, the present disclosure provides a blood pressure measurement device, the blood pressure measurement device including an air pressure sensor, a piezoelectric sensor and a control module;
the air pressure sensor being configured to collect pressure analog signals which are used to characterize air pressure inside an air bladder;
the piezoelectric sensor being configured to be fixed in a cuff and used to collect brachial artery pulse signals; and
the control module being configured to obtain the pressure analog signals and the brachial artery pulse signals, to recognize the brachial artery pulse signals to obtain Korotkoff sound signals, and to obtain Korotkoff sound data according to the Korotkoff sound signals, and the control module being further configured to process the pressure analog signals and the Korotkoff sound signals to obtain blood pressure data, the blood pressure data include air pressure and blood pressure values during air bladder pressure deflation process, the blood pressure values being systolic pressure and diastolic pressure, and the control module being further configured to respond to a playback operation to perform synchronous playback of the Korotkoff sounds and the air pressure according to Korotkoff sound data and air pressure data, and simultaneously displays blood pressure values. The air pressure sensor and the piezoelectric sensor are respectively connected with the control module for communication.

The blood pressure measurement device provided by this embodiment, via mutual cooperation of the air pressure sensor, the piezoelectric sensor and the control module, can accurately recognize a Korotkoff sound signal from the brachial artery pulse signals, thereby to accurately determine Korotkoff sound data and blood pressure data corresponding to the Korotkoff sound data, and respond to a playback operation, to perform synchronous playback of the Korotkoff sounds and the air pressure, and simultaneously display the systolic pressure and the diastolic pressure, which provides original measurement data for determining a blood pressure value, and helps to improve the accuracy of blood pressure measurement.

In a third aspect, the present disclosure provides a blood pressure measuring method with Korotkoff sounds recognition and playback function, the method including:
obtaining pressure analog signals and brachial artery pulse signals in a process in which air pressure inside an air bladder deflates;
recognizing the brachial artery pulse signals to obtain Korotkoff sound signals;
processing the pressure analog signals to obtain air pressure in the process in which the air pressure inside the air bladder deflates;
processing the pressure analog signals and the Korotkoff sound signals to obtain systolic pressure data and diastolic pressure data; and
in response to a playback operation, synchronously playing back the process in which the air pressure inside the air bladder deflates, a process from the appearance to the disappearance of the Korotkoff sounds, and displaying the systolic pressure data and the diastolic pressure data.

In a fourth aspect, the present disclosure further provides a blood pressure measuring method, the method including:
obtaining pressure analog signals and brachial artery pulse signals;
recognizing the brachial artery pulse signals to obtain Korotkoff sound signals, and obtaining Korotkoff sound data according to the Korotkoff sound signals;
processing the pressure analog signals and the Korotkoff sound signals to obtain blood pressure data, the blood pressure data includes air pressure and blood pressure values of an air bladder pressure deflation process, the blood pressure value being systolic pressure and diastolic pressure; and
in response to a playback operation, performing synchronous playback of Korotkoff sounds and air pressure according to the Korotkoff sound data and the air pressure data, and displaying the blood pressure values.

In the blood pressure measuring method provided by this embodiment, the Korotkoff sound signals are recognized according to the brachial artery pulse signals, then blood pressure data are obtained according to the Korotkoff sound signals and the pressure analog signals inside the air bladder, the corresponding air pressure when a Korotkoff sound appears is recognized as a systolic pressure, and the corresponding air pressure when a Korotkoff sound disappears is recognized as a diastolic pressure, a measurement result is ensured to be accurate, correlation among the Korotkoff sounds, the blood pressure data and the measurement time during measurement is established, and the Korotkoff sounds, the blood pressure data, the measurement time and the correlation among them are stored as one-time blood pressure measurement data, in response to a playback operation, the Korotkoff sounds and the air pressure in the air bladder pressure deflation process are played back synchronously, the systolic pressure is displayed when the Korotkoff sound appear, and the diastolic pressure is displayed when the Korotkoff sound disappear, so as to facilitate a user to verify whether blood pressure measurement is accurate according to the process from the appearance to the disappearance of the Korotkoff sounds.

In a fifth aspect, the present disclosure provides an electronic device, including a memory and a processor, the memory and the processor communicatively connect with each other, a computer instruction is stored in the memory, and the processor performs the method in the above third aspect or fourth aspect or any one corresponding implementation by executing the computer instruction.

### BRIEF DESCRIPTION OF DRAWINGS

In order to describe a technical solution in a specific implementation of the present disclosure or in a related technology more clearly, the drawings required for use in the specific implementation or the related technology are briefly introduced below. Obviously, the drawings in the following description are some implementations of the present disclosure. Persons skilled in the art may further obtain other drawings according to these drawings under the premise that they do not pay inventive labor.

FIG. 1 is a structure schematic diagram of a blood pressure measurement device according to the embodiments of the present disclosure;

FIG. 2 is a structure schematic diagram of an air bladder according to the embodiments of the present disclosure;

FIG. 3 is a structure schematic diagram of another blood pressure measurement device according to the embodiments of the present disclosure;

FIG. 4 is a schematic diagram of a blood pressure measurement scenario according to the embodiments of the present disclosure;

FIG. 5 is a curve schematic diagram of Korotkoff sound signals according to the embodiments of the present disclosure;

FIG. 6 is another curve schematic diagram of Korotkoff sound signals according to the embodiments of the present disclosure;

FIG. 7 is a schematic diagram of a frequency spectrogram graph of a Korotkoff sound signals according to the embodiments of the present disclosure;

FIG. 8 is a schematic diagram of a Korotkoff digital signals processing procedure according to the embodiments of the present disclosure;

FIG. 9 is a schematic diagram of a display interface according to the embodiments of the present disclosure;

FIG. 10 is a flow schematic diagram of a blood pressure measuring method according to the embodiments of the present disclosure;

FIG. 11 is a flow schematic diagram of Korotkoff sounds playback and blood pressure playback according to the embodiments of the present disclosure;

FIG. 12 is a flow schematic diagram of another blood pressure measuring method according to the embodiments of the present disclosure;

FIG. 13 is a flow schematic diagram of a further blood pressure measuring method according to the embodiments of the present disclosure;

FIG. 14 is a hardware structure schematic diagram of an electronic device according to the embodiments of the present disclosure.

### REFERENCE SIGNS

101, air pressure sensor; 102, piezoelectric sensor; 1021, first piezoelectric plate; 1022, second piezoelectric plate; 103, control module; 104, storage module; 105, audio output module; 106, display module; 107, cuff; 1071, fixing element; 108, air bladder; 1081, air nozzle; 109, air hose; 110, air pump; 200, main unit; 201, first volume button; 202, second volume button; 203, control button; 204, icon display button; 205, play button; 310, processor; 320, memory; 330, communication interface.

### DETAILED DESCRIPTION

In order to make the purpose, technical solutions and advantages of the embodiments of the present disclosure clearer, the technical solutions in the embodiments of the present disclosure are described clearly and completely with reference to the drawings in the embodiments of the present disclosure, obviously the described embodiments are some embodiments of the present disclosure, not all embodiments. According to the embodiments in the present disclosure, all other embodiments obtained by persons skilled in the art without paying inventive labor fall into the scope protected by the present disclosure.

Blood pressure is pressure of blood flowing in blood vessels to side walls of the blood vessels, is a force that pushes blood to flow in the blood vessels, and is an important parameter reflecting fundamental life signs of a human body. Blood pressure may provide a basis for doctors to assess a patient's physical condition, for example, may be used to determine whether the patient has high blood pressure, cardiovascular disease or kidney disease, so blood pressure measurement is crucial.

For the non-invasive blood pressure measurement technology, the most recognized and accurate method is a Korotkoff sound method, which judges a blood pressure value via monitored sounds of blood flowing in the brachial artery during the deflation of the cuff's air bladder. However, there are many limitations in the process of blood pressure measurement by using the Korotkoff sound method, such as relying on hearing sense and subjective judgment of a medical staff and being susceptible to an impact of environmental noise, which may lead to an error in a measured blood pressure and affect a diagnosis result for a disease by the medical staff.

In view of this, the present disclosure provides a blood pressure measurement device with a Korotkoff sounds recognition and playback function, i.e., a sphygmomanometer with a Korotkoff sounds recognition and playback function, via mutual cooperation of the air pressure sensor, the piezoelectric sensor and the control module, can accurately recognize a Korotkoff sound signal from the brachial artery pulse signals, thereby to accurately determine Korotkoff sound data and air pressure data corresponding to the Korotkoff sound data, which provides original measurement data for determining a blood pressure value, and helps to improve the accuracy of blood pressure measurement. The Korotkoff sound is the sound of a blood flow of a brachial artery rushing against a blood vessel wall when an air bladder transitions from pressurization during inflation to depressurization during deflation, the first sound of a blood flow impacting a blood vessel wall is first Korotkoff sound in a blood pressure measurement process, the pressure inside the air bladder corresponding to the first Korotkoff sound is a systolic pressure of a blood pressure value, i.e., a high pressure, and the sound when the sound of a blood flow impacting a blood vessel wall weakens or disappears is last Korotkoff sound in the blood pressure measurement process, the pressure inside the air bladder corresponding to the last Korotkoff sound is a diastolic pressure of the blood pressure value, i.e., a low pressure.

In the present disclosure, the blood pressure value is obtained according to Korotkoff sounds and the pressure inside the air bladder, the blood pressure value is the systolic pressure and the diastolic pressure. And, the blood pressure measurement device of the present disclosure, in response to a playback operation of a user, plays back the Korotkoff sounds for determining a blood pressure in a blood pressure measurement process, air pressure in an air bladder depressurization process, and displays the blood pressure value, which specifically is: in response to a playback operation, synchronously playing back the Korotkoff sounds and the air pressure and simultaneously displaying the systolic pressure and the diastolic pressure, characterizing playback of the Korotkoff sounds by sound playing or the pulsation of a heartbeat graphic symbol on a display screen, characterizing playback of the air bladder depressurization process via digital changes displayed on the display screen, and displaying blood pressure values by combining a digit with words “systolic pressure” or “high pressure,” “diastolic pressure” or “low pressure”.

Embodiments provided by the present disclosure can recognize Korotkoff sounds to obtain blood pressure values and play back the Korotkoff sounds to judge whether the blood pressure values are accurate, so as to facilitate a user to verify the accuracy of a blood pressure measurement result by analyzing the Korotkoff sounds.

The blood pressure measurement device provided by the present disclosure is described in detail below with reference to the drawings.

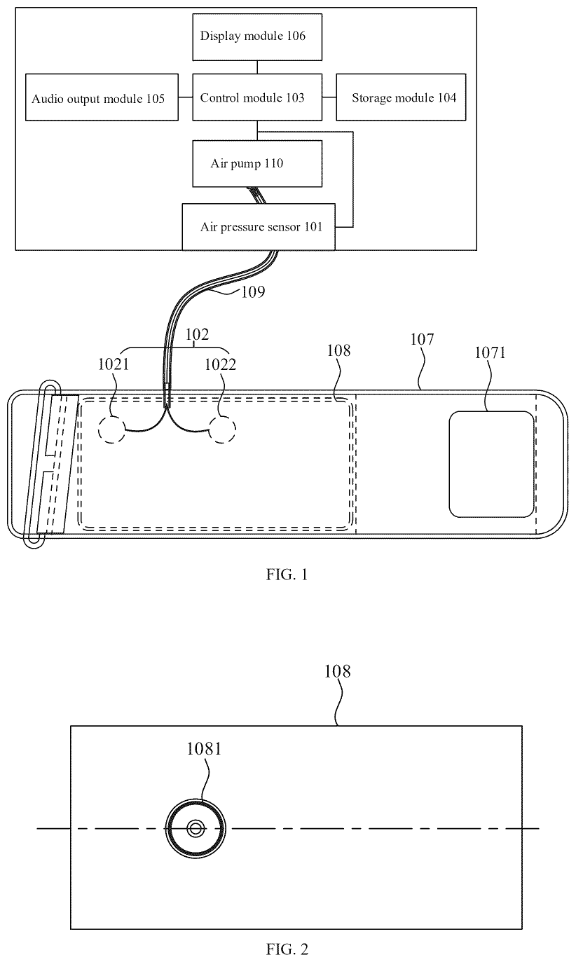

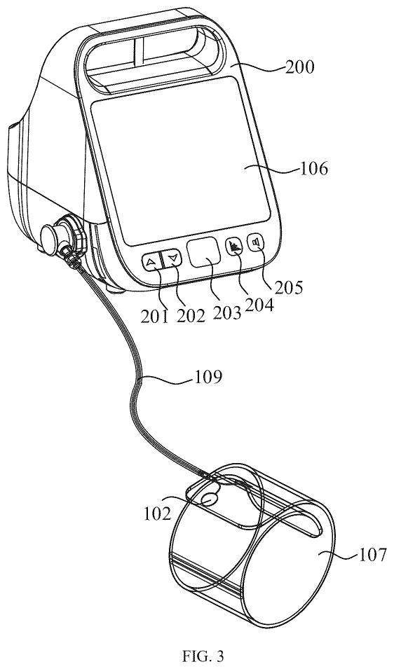

As shown in FIG. 1 and FIG. 3, the blood pressure measurement device includes but is not limited to a main unit 200, an air pressure sensor 101, a piezoelectric sensor 102, a control module 103, a storage module 104, an audio output module 105 and a display module 106.

The piezoelectric sensor 102 is fixed in a cuff 107, a fixing method includes but is not limited to adhesion or snap-fastening, etc. The air pressure sensor 101 is provided in the main unit 200, the air pressure sensor 101 and the piezoelectric sensor 102 are communicatively connected with the control module 103. The piezoelectric sensor 102 is a vibration sensor, and another vibration sensor, i.e., a microphone, may further be used in this embodiment.

The air pressure sensor 101 is configured to collect pressure analog signals and transmit the pressure analog signals to the control module 103, the pressure analog signals being used to characterize air pressure inside an air bladder of the cuff 107. The piezoelectric sensor 102 is configured to collect a brachial artery pulse signals and transmit the brachial artery pulse signals to the control module 103, the brachial artery pulse signals being used to represent a sound of blood flowing of a brachial artery in a blood pressure measurement process.

As an embodiment, the control module 103 is configured to control the air pressure inside the air bladder and recognize Korotkoff sounds in the brachial artery pulse signals collected by the vibration sensor, and obtain systolic pressure data and diastolic pressure data according to the Korotkoff sounds and the air pressure inside the air bladder; and the control module 103 is further configured to play back and display, in response to a playback operation, air pressure deflation process inside the air bladder, a process from the appearance to the disappearance of the Korotkoff sounds, and the systolic pressure data and the diastolic pressure data. The vibration sensor is a piezoelectric sensor 102 or a microphone. And, playback of the air pressure deflation process inside the air bladder is displayed digitally, and playback of the process from the appearance to the disappearance of the Korotkoff sounds is performed via the audio output module 105 i.e., a sound player and/or via pulsation of a heartbeat graphic symbol on the display module 106 i.e., a display screen. The control module is further configured to store blood pressure measurement data in the local storage module 104, and transmit the blood pressure measurement data to an external device such as a mobile phone or a computer device in a wireless or wired way for storage, and perform synchronous playback of the Korotkoff sounds and pressure and display a blood pressure value according to the playback operation.

As another embodiment, the control module 103 is configured to recognize brachial artery pulse signals to obtain Korotkoff sound signals, and to process pressure analog signals and the Korotkoff sound signals to obtain blood pressure data, the blood pressure data including blood pressure values serving as a blood pressure measurement result, that is, a systolic pressure recognized as a high pressure and a diastolic pressure recognized as a low pressure. The control module 103 is further configured to obtain Korotkoff sound data according to the Korotkoff sound signals. The control module 103 further stores the blood pressure data and the Korotkoff sound data simultaneously in the storage module 104, and is configured to perform synchronous playback of Korotkoff sounds playback and air pressure playback in response to a playback operation, and display blood pressure values at the same time or display the blood pressure value after the playback ends, and establish a correspondence relationship between the Korotkoff sound data and the air pressure according to a playback time period, that is, play back the Korotkoff sound data corresponding to the air pressure at the same time of playing back the air pressure, and play back the air pressure corresponding to the Korotkoff sound data at the same time of playing back the Korotkoff sound data. That is, in response to the playback operation, the control module 103 plays back the Korotkoff sound data and the blood pressure data synchronously, that is, plays back the Korotkoff sounds, the air pressure in an air bladder pressure deflation process and the blood pressure value synchronously.

The present disclosure does not limit a type and a specific position of the air pressure sensor 101, as long as air pressure in the air bladder 108 of the cuff 107 can be collected during blood pressure measurement. For example, the air pressure sensor 101 may be a piezoelectric air pressure sensor, a capacitive air pressure sensor, or a strain-gauge air pressure sensor, etc.

Illustratively, as shown in FIG. 1 and FIG. 2, the cuff 107 has an accommodation space, the air bladder 108 for storing air is provided in the accommodation space, the air bladder 108 is provided with an air nozzle 1081, the air bladder 108 is connected to an air pump 110 via the air nozzle 1081 and an air hose 109, and the air pump 110 is connected to the control module 103. In this embodiment, the air pressure sensor 101 may be provided in an end of the air hose 109 near an end of the air pump 110, the pressure of the air bladder 108 is transmitted to the air pressure sensor 101 via the air nozzle 1081 and the air hose 109, and a specific position of the air pressure sensor 101 on the air hose 109 may be determined by a designer according to experience.

Optionally, an air valve (not shown in the figure) is provided at one end of the air hose 109 near the control air pump 110, the air valve is connected with the control module 103, the control module 103 may adjust a speed of pressurizing or depressurizing the air bladder 108 by controlling an opening and closing degree of the air valve, so as to control air pressure in the air bladder 108, slow and steady depressurization helps to collect the Korotkoff sounds more accurately.

Specifically, the opening and closing degree is used to represent a degree of opening or closing of the air valve, the opening and closing degree may be expressed by a proportion value or angle value. If the opening and closing degree is defined to represent a degree of opening, 100% represents that the air valve is fully opened. At this point, the greater the opening and closing degree (the closer to 100%), the greater a gas flow rate and the faster an air bladder pressurization or depressurization speed.

Embodiments of the present disclosure do not limit a specific position of the piezoelectric sensor 102, as long as Korotkoff sounds can be collected during blood pressure measurement. The piezoelectric sensor 102 may be fixedly provided at a lower side of the air bladder 108 within the cuff 107 to position the piezoelectric sensor 102 at a brachial artery position of an arm during blood pressure measurement. Two piezoelectric sensors may be set, respectively corresponding to a brachial artery position of the left arm and a brachial artery position of the right arm. The lower side of the air bladder 108 refers to a side of the air bladder 108 that faces a user's skin when a blood pressure is measured, i.e., an inner wall of the air bladder. Illustratively, the piezoelectric sensor 102 may further be a microphone. Both the piezoelectric sensor and the microphone are vibration sensors.

The control module 103 is further communicatively connected with the storage module 104, the audio output module 105 and the display module 106 respectively, the control module 103 is configured to obtain pressure analog signals and brachial artery pulse signals, and to recognize the brachial artery pulse signals to obtain Korotkoff sound signals and obtain Korotkoff sound data according to the Korotkoff sound signals, the control module 103 is further configured to process the pressure analog signals and the Korotkoff sound signals to obtain blood pressure data, in which the blood pressure data includes air pressure and blood pressure values of an air bladder pressure deflation process, the blood pressure values being systolic pressure and diastolic pressure. The control module 103 is further configured to store the blood pressure data and the Korotkoff sound data to the storage module simultaneously, and to perform synchronous playback of the Korotkoff sound data and the blood pressure data in response to a playback operation, to control the audio output module 105 to perform Korotkoff sounds playback of the Korotkoff sound data read from the storage module 104, and control the display module 106 to perform air pressure playback and display blood pressure values by reading the blood pressure data corresponding to the Korotkoff sound data from the storage module 104. That is, when the audio output module 105 plays the Korotkoff sounds, the display module 106 displays pressure in the air bladder 108 when the Korotkoff sounds appears. The control module 103 is further configured to correlate a measuring time, air pressure and blood pressure values included by the blood pressure measurement data during blood pressure measurement with the Korotkoff sound data and store the correlation to the storage module, and in response to a playback operation, play back air pressure deflation process in the air bladder and a process from the appearance to the disappearance of the Korotkoff sounds, and display blood pressure values at the same time.

Specifically, the control module 103 first recognizes brachial artery pulse signals to obtain Korotkoff sound signals, selects pressure analog signals according to the Korotkoff sound signals, retains the pressure analog signals corresponding to an collection moment of the Korotkoff sound signals, converts the Korotkoff sound signals and the pressure analog signals corresponding to the Korotkoff sound signals to obtain blood pressure data, the blood pressure data includes air pressure of an air bladder depressurization process and a blood pressure value, a format of the blood pressure data is adapted to a format that can be recognized by the display module, Korotkoff sound data is obtained by converting the Korotkoff sound signals, a format of the Korotkoff sound data is adapted to a format that can be recognized by the audio output module.

That is to say, after the control module 103 obtains the pressure analog signals from the air pressure sensor 101 and the brachial artery pulse signals from the piezoelectric sensor 102, the control module 103 processes the pressure analog signals to obtain pressure in the air bladder, recognizes the brachial artery pulse signals to obtain Korotkoff sound signals, then selects a pressure analog signals according to the Korotkoff sound signals, performs conversion processing on the selected pressure analog signals and the Korotkoff sound signals to be blood pressure data that can be recognized by the display module 106, displays a blood pressure measurement result, i.e., a systolic pressure and a diastolic pressure, converts the Korotkoff sound signals into Korotkoff sound data that can be recognized by the audio output module 105, and stores the blood pressure data and the Korotkoff sound data simultaneously to the storage module 104.

After that, if the user performs the playback operation (for example pressing a preset button with a preset action), the playback operation further includes that the user selects blood pressure measurement data needing to be played back through a function button on a blood pressure measurement device, then presses the preset button with the preset action to start the playback operation. In response to the playback operation, the control module 103 synchronously plays back a pressure in an air bladder depressurization process, Korotkoff sounds and blood pressure values, reads Korotkoff sound data from the storage module 104, and controls the audio output module 105 to play the Korotkoff sounds appearing during blood pressure measurement. At the same time, the control module 103 further reads blood pressure data from the storage module 104, and controls the display module 106 to simultaneously display air pressure corresponding to the Korotkoff sounds played during the blood pressure measurement, and simultaneously display the blood pressure values. That is, when the user performs the playback operation, the blood pressure measurement device plays back Korotkoff sound data and air pressure data synchronously and displays the blood pressure values. The process of conversion processing is introduced in the following text, and is not described in details here.

What needs illustration is that in order to ensure synchronous collection of the Korotkoff sounds and the air pressure, a collection start moment of the air pressure sensor 101 is the same as a collection start moment of the piezoelectric sensor 102, the collection start moment of the air pressure sensor 101 refers to time when the air pressure sensor 101 starts to collect pressure analog signals, the collection start moment of the piezoelectric sensor 102 refers to time when the piezoelectric sensor 102 starts to collect a brachial artery pulse signals.

Illustratively, the collection start time of the air pressure sensor 101 and the collection start time of the piezoelectric sensor 102 may be synchronized via a timer or other timing device.

Illustratively, the control module 103 in this embodiment may be a Central Processing Unit (CPU), a Microcontroller Unit (MCU), or a programmable logic device that implements the above functions, and so on. The storage module 104 may be a common storage medium, such as a Secure Digital Memory Card (SD), a flash memory or a Random Access Memory (RAM), etc. The audio output module 105 may be a loudspeaker or an earphone, etc. The display module 106 may be a liquid crystal display, a Light Emitting Diode Display (LED) display screen or a touch screen, etc.

It should be understood that, as shown in FIG. 1, the cuff 107 is further provided with a fixing element 1071 which includes but is not limited to a strap or a Velcro, etc.

Specifically, as shown in FIG. 3, the air pressure sensor 101, the air pump 110, the control module 103, the storage module 104, the audio output module 105 and the display module 106 may all be integrated on the main unit 200.

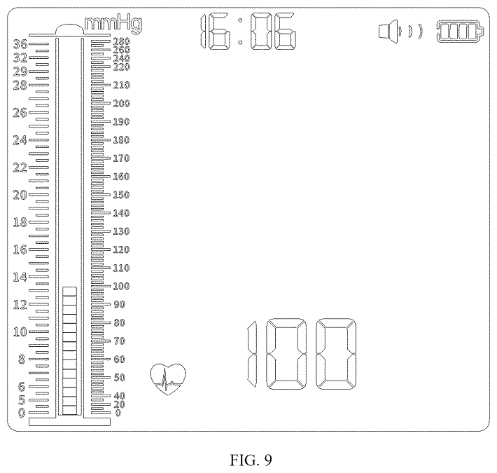

The process of blood pressure measurement using the blood pressure measurement apparatus in the first embodiment and the second embodiment provided by the present disclosure is described below by taking FIGS. 1, 3 and 4 as examples. The air pump 110, the control module 103, the storage module 104, the audio output module 105 and the display module 106 are integrated in the main unit 200 as shown in FIG. 3. The display module 106 is a display screen of the main unit 200, and a heartbeat graphic symbol is displayed on the display screen, as shown in FIG. 9, in a test process, the heartbeat graphic symbol displays in a pulsation manner a frequency and process in which the KororKoff sound appears, as auscultated using an auscultatory method, and in the process of KororKoff sounds playback, the heartbeat graphic symbol displays in a pulsation manner a frequency and process of KororKoff sounds playback. Of course, the playback of the KororKoff sounds may further be played by the audio output module 105.

When a medical staff measures a patient's blood pressure or a user performs blood pressure measurement by himself/herself, first, as shown in FIG. 3, the cuff 107 is crimped and fixed on the user's arm via the fixing element 1071, so that the piezoelectric sensor 102 is located at a brachial artery position of the arm. The user then initiates blood pressure measurement, and the control module 103 in the main unit 200, in response to a measurement operation, controls the air pump 110 to inflate the air bladder 108 via the air hose 109 and the air nozzle 1081 to increase the pressure of the air bladder 108 until the air pressure in the air bladder can block blood flowing of an artery blood vessel. After that, the control module 103 controls the air pump 110 to deflate the air bladder 108 via the air hose 109 and the air nozzle 1081, so that the pressure inside the air bladder 108 slowly drops. In this process, the air pressure sensor 101 collects the air pressure in the air bladder during deflation in real time, and transmits pressure analog signals characterizing the air pressure in the air bladder to the control module 103, the piezoelectric sensor 102 collects brachial artery pulse signals in real time, and transmits the brachial artery pulse signals to the control module 103.

After receiving the pressure analog signals and the brachial artery pulse signals, the control module 103 recognizes the brachial artery pulse signals to obtain Korotkoff sound signals, processes the pressure analog signals to obtain air pressure in the air bladder, recognizes the corresponding air pressure in the air bladder when the first Korotkoff sound appears as a systolic pressure, and recognizes the corresponding air pressure in the air bladder when the Korotkoff sound disappears or weakens as a diastolic pressure. Moreover, the Korotkoff sounds, the air pressure and the blood pressure value in the blood pressure measurement process are stored locally in real time or are transmitted to an external device for storage after the blood pressure measurement is completed. In response to the playback operation, the control module 103 synchronously plays back the Korotkoff sounds and the air pressure in the blood pressure measurement process, displays a systolic pressure when the Korotkoff sounds appears, displays a diastolic pressure when the Korotkoff sounds disappears or weakens, or displays the systolic pressure and the diastolic pressure after playback of Korotkoff sounds and pressure ends. Embodiments provided by the present disclosure can not only recognize the Korotkoff sounds to perform accurate blood pressure measurement, but also can play back the air pressure and the Korotkoff sounds in the blood pressure measurement process, so as to facilitate a user to verify the accuracy of blood pressure measurement by analyzing a change in the Korotkoff sounds.

As another embodiment, after receiving the pressure analog signals and the brachial artery pulse signals, the control module 103 may further recognize the brachial artery pulse signals to obtain a Korotkoff sound signals, and obtain Korotkoff sound data according to the Korotkoff sound signals, then process the pressure analog signals and Korotkoff sound signals to obtain blood pressure data which includes air pressure data and a blood pressure value recognized according to the Korotkoff sounds, then store the blood pressure data and the Korotkoff sound data in the storage module 104, and may further correlate the blood pressure data, the Korotkoff sound data and a corresponding measurement time as blood pressure measurement data and store the same in the storage module 104 at the same time. When the user performs the playback operation, in response to the playback operation, the control module 103 plays back the Korotkoff sounds in the blood pressure measurement process, the air pressure in the air bladder depressurization process and a blood pressure value, specifically: the control module 103 reads the Korotkoff sound data from the storage module 104 and controls the audio output module 105 to play the Korotkoff sounds or to display the Korotkoff sounds by the pulsation of the heartbeat graphic symbol on the display screen, or play back the Korotkoff sounds at the same time in a manner that the audio output module 105 plays the Korotkoff sounds or displays the Korotkoff sounds by the pulsation of the heartbeat graphic symbol on the display screen, meanwhile, the control module 103 further reads blood pressure data corresponding to the Korotkoff sound data from the storage module 104, and controls the display module 106 to digitally display air pressure values in the blood pressure data corresponding to the played Korotkoff sounds and simultaneously display blood pressure values. The control module 103 sets a recognition threshold of the Korotkoff sound signals according to a result of comparison between an average arterial signal intensity of the brachial artery pulse signals within a preset time period and a preset threshold, and is used to recognize Korotkoff sound signals according to the recognition threshold, and determines the corresponding arterial intensity signal whose arterial signal intensity is greater than the recognition threshold as a Korotkoff sound signal.

The blood pressure measurement device provided by this embodiment, via mutual cooperation of the air pressure sensor 101, the piezoelectric sensor 102 and the control module 103, may accurately recognize a Korotkoff sound signals from the brachial artery pulse signals, thereby to accurately determine Korotkoff sound data and air pressure corresponding to the Korotkoff sound data, and recognize blood pressure values according to the Korotkoff sounds and the air pressure, and when responding to the playback operation, simultaneously play back the Korotkoff sounds and the air pressure of the air pressure deflation process in the air bladder, and simultaneously display the blood pressure values. This embodiment provides original measurement data for determining the blood pressure values, which is helpful to improve the accuracy of blood pressure measurement, and by playing back the Korotkoff sounds and the air pressure in the air bladder, facilitates a user to observe a time of appearance and disappearance of the Korotkoff sounds, and judge the blood pressure values by combining the air pressure that is played back, and provides a self-proving scheme for the accuracy of a blood pressure measurement result.

At the same time, by providing the storage module 104, the audio output module 105 and the display module 106, the blood pressure measurement device can not only record and store the Korotkoff sounds and the blood pressure values in the blood pressure measurement process in real time, ensuring integrity and accuracy of data, but also can play back at any time the Korotkoff sounds in the measurement process, the air pressure of the air pressure depressurization process in the air bladder, and display the blood pressure values corresponding to the Korotkoff sounds in the measurement process, which is convenient for medical staff to view and analyze collected data many times, thereby to determine a user's blood pressure more accurately, which helps medical staff to judge more accurately whether there is a phenomenon such as arrhythmia or atrial fibrillation.

Specifically, in blood pressure measurement process, the air pressure sensor 101 may collect pressure analog signals at a pressure sampling frequency f1 to obtain a plurality of pressure analog signals. The piezoelectric sensor 102 may collect brachial artery pulse signals at a piezoelectric sampling frequency to obtain a plurality of brachial artery pulse signals. After obtaining the plurality of pressure analog signals and the plurality of brachial artery pulse signals, the control module 103 may recognize multiple the plurality of brachial artery pulse signals to obtain a plurality of Korotkoff sound signals, and process the plurality of pressure analog signals to obtain multiple pieces of pressure data, i.e., air pressure data in the air bladder, then, process the plurality of pressure analog signals and the plurality of Korotkoff sound signals to obtain multiple pieces of blood pressure data, and process the plurality of Korotkoff sound signals to obtain multiple pieces of Korotkoff sound data, the blood pressure data here includes air pressure data. Moreover, the multiple pieces of pressure data are provided with air pressure playback sequence number according to a collection moment, and multiple pieces of Korotkoff sound data are provided with audio playback sequence number according to the collection moment. When Korotkoff sounds are played back, the multiple pieces of Korotkoff sound data are played according to an audio playing frequency f2.

In order to ensure that a played Korotkoff sounds corresponds to a displayed blood pressure, the control module 103 first determines a playback data segment and a playback time period according to the pressure analog signals and Korotkoff sound signals. The playback data segment includes a data segment of Korotkoff sounds playback and a data segment of blood pressure playback, and the playback time period is a time period in which the Korotkoff sound data and the blood pressure data are played back simultaneously.

Specifically, the data segment of the Korotkoff sounds playback includes Korotkoff sound data from the Nth second before a start moment of the Korotkoff sound data to the Mth second after a disappearance moment of the Korotkoff sound data, the data segment of the air pressure playback is air pressure data corresponding to a data segment of the Korotkoff sounds playback, the playback time period is consistent with a duration of the playback data segment, N≥1 and M≥1. The data segment of the Korotkoff sounds playback may further include noise data from the Nth second before a start moment of the Korotkoff sound data to the Mth second after a disappearance moment of the Korotkoff sound data.

Playback time period T3 is a difference between a playback end moment T2 of Korotkoff sounds playback and a playback start moment T1 of Korotkoff sounds playback, that is, T3=T2−T1. The playback end moment is the Mth second after a disappearance moment of the Korotkoff sound data. The playback start moment is the Nth second before a start moment of the Korotkoff sound data.

By taking N=1 and M=1 as an example, the data segment of Korotkoff sounds playback includes Korotkoff sound data from 1 s before an appearance moment of the first Korotkoff sound to 1 s after an appearance moment of the last Korotkoff sound. The data segment of air pressure playback is all the air pressure data in a time period corresponding to the data segment of Korotkoff sounds playback. At this point, the playback time period may be a difference between 1 s before an appearance moment of the first Korotkoff sound and 1 s after an appearance moment of the last Korotkoff sound.

After the playback time period is determined, the control module 103 is further used to determine audio playback sequence number of the Korotkoff sounds playback according to the playback time period and an audio playback frequency of the Korotkoff sounds, and determine air pressure playback sequence number of the air pressure playback according to the playback time period and a pressure sampling frequency of the air pressure. The control module 103 is further used to read the Korotkoff sound data from the storage module according to the audio playback sequence number, and read the air pressure data corresponding to the Korotkoff sound data from the storage module according to the air pressure playback sequence number. A corresponding relationship between Korotkoff sound data and air pressure data at the same moment is established according to the playback time period by the audio playback sequence number and the air pressure playback sequence number, and in the process of playing back the Korotkoff sounds, the air pressure value corresponding to the first Korotkoff sound when the Korotkoff sound appears is recognized as a systolic pressure for display, and the corresponding air pressure value when the Korotkoff sound is weakened or disappears is recognized as a diastolic pressure for display.

The audio playback sequence number may be the product of the playback time period T3 and the audio playing frequency f2, i.e., the audio playback sequence number is (T3×f2). The air pressure playback sequence number may be the product of the playback time period T3 and the pressure sampling frequency f1, i.e., the air pressure playback sequence number is (T3×f1).

The above mentioned process of recognizing the brachial artery pulse signals to obtain Korotkoff sound signals is described in details below with reference to the drawings.

Illustratively, the control module 103 sets a recognition threshold of the Korotkoff sound signals according to a comparison result between an average arterial signal intensity of the brachial artery pulse signals within a preset time period and a preset threshold, and is used to recognize Korotkoff sound signals according to the recognition threshold.

Specifically, after obtaining the brachial artery pulse signals, the control module 103 performs band-pass filtering processing on the brachial artery pulse signals within a preset frequency range to remove an interference signal to obtain an arterial intensity signal. Then, the control module 103 obtains a numerical difference between a maximum value and a minimum value of the arterial intensity signal within a preset collection time period for each instance, and takes the numerical difference as an arterial signal intensity for each instance; after that, the control module 103 obtains an average value of the arterial signal intensity across all instances to obtain a mean value of the arterial signal intensity, and sets a recognition threshold of Korotkoff sound signals according to comparison result among the average arterial signal intensity, a first preset threshold and a second preset threshold.

After that, the control module 103 determines whether the intensity of the arterial intensity signal for each instance (that is, arterial signal intensity) is greater than the recognition threshold. If the arterial signal intensity of the arterial intensity signal is greater than the recognition threshold, the arterial intensity signal is determined as a Korotkoff sound signal. That is, the control module 103 determines the brachial artery pulse signal corresponding to the arterial intensity signal whose arterial signal strength is greater than the recognition threshold as a Korotkoff sound signal.

Illustratively, after the average arterial signal intensity is determined, the recognition threshold of the Korotkoff sound signals is set to be a first recognition threshold when the average arterial signal intensity is less than the first preset threshold; and the second preset threshold of the Korotkoff sound signals is set to be P times the average arterial signal intensity when the average arterial signal intensity is greater than the second preset threshold, and a third recognition threshold of the Korotkoff sound signals is set to be 2P times the average arterial signal intensity when the average arterial signal intensity is between the first preset threshold and the second preset threshold.

Specifically, the average arterial signal intensity between the first preset threshold and the second preset threshold means that the average arterial signal intensity is greater than or equal to the first preset threshold, and the average arterial signal intensity is less than or equal to the second preset threshold.

P>0, the preset frequency range, the preset collection time period, the first preset threshold, the second preset threshold, the first recognition threshold, and P may all be empirical values and may be adjusted according to actual needs. For example, the preset frequency range may be [20 Hz, 120 Hz], the preset collection time period may be 1 s or 2 s, etc., the first preset threshold may be 0.005, the second preset threshold may be 0.008, the first recognition threshold may be 0.015, and P may be 1.5.

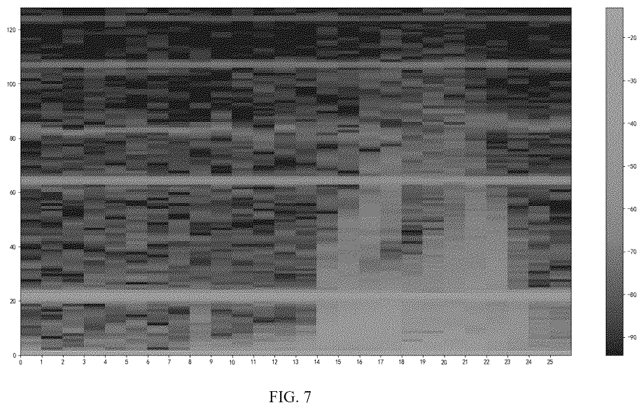

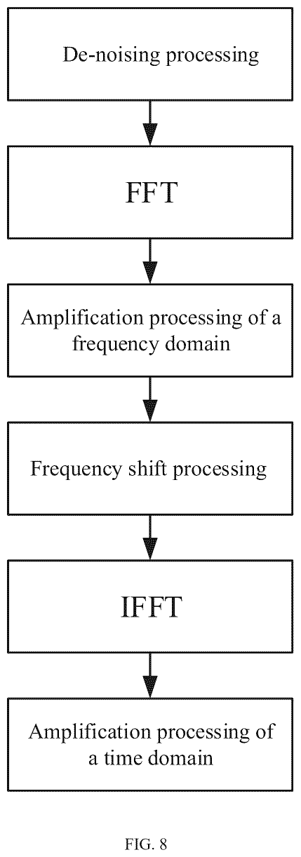

By taking “the preset frequency range is [20 Hz, 120 Hz], the preset collection time period is 1 s or 2 s, etc., the first preset threshold is 0.005, the second preset threshold is 0.008, the first recognition threshold is 0.015, and P is 1.5” as an example, the process of recognizing the brachial artery pulse signals to obtain Korotkoff sound signals is described. Specifically, after the brachial artery pulse signals is obtained, band pass filtering with [20 Hz, 120 Hz] is performed on the brachial artery pulse signals to remove interference signals and obtain an arterial intensity signal, the arterial intensity signal may be as shown in FIG. 5 or FIG. 6. After the arterial intensity signal is determined, maximum and minimum values of arterial intensity signal in data within 1 s for each instance are determined, and a numerical difference between the maximum and minimum values is taken as an arterial signal intensity for each instance. After arterial signal intensities of all the arterial intensity signals are obtained, an average value of the arterial signal intensities of all times is determined as an average arterial signal intensity.

A recognition threshold is then determined, specifically, when the average arterial signal intensity is less than 0.005, the recognition threshold of the Korotkoff sound signals is a first recognition threshold, i.e., 0.015; when the average arterial signal intensity is greater than 0.008, the recognition threshold of Korotkoff sound signals is a second recognition threshold, i.e., 1.5 times the average arterial signal intensity, and when the average arterial signal intensity is between intervals [0.005, 0.008], the recognition threshold is a third recognition threshold, i.e., 3 times the average arterial signal intensity.

After that, whether the intensity of the arterial intensity signal each time is greater than the recognition threshold is determined. If the arterial signal intensity is greater than the recognition threshold, the arterial intensity signal is determined as Korotkoff sound signals, a spectrogram diagram of the Korotkoff sound signals may be as shown in FIG. 7.

Specifically, the transverse coordinate in FIG. 5 and FIG. 6 represents time, which may be in second(s), and the vertical coordinate represents an amplitude of the Korotkoff sound signals, which may be in volt(s) (V). FIG. 7 shows energy distribution of the Korotkoff sound signals at different frequencies.

In some optional implementations, the control module 103 is further used to determine a blood pressure measurement result according to the blood pressure data and the Korotkoff sound data after the blood pressure data and the Korotkoff sound data are determined, and is used to control the display module 106 to display the blood pressure measurement result, and the control module 103 responds to a playback operation after the display module 106 displays the blood pressure measurement result.

The blood pressure measurement result includes a low pressure signal and a high pressure signal, to achieve measurement of a high blood pressure and a low blood pressure. The high pressure signal refers to a blood pressure value corresponding to the first Korotkoff sound during blood pressure measurement, and the low pressure signal refers to a blood pressure value corresponding to disappearance or change of Korotkoff sounds during blood pressure measurement. Therefore, the blood pressure data in this embodiment includes air pressure in an air bladder depressurization process, and blood pressure values as a blood pressure measurement result.

In some optional implementations, the piezoelectric sensor 102 may communicate with the control module 103 via a connection line built into the air hose 109. The piezoelectric sensor 102 includes a first piezoelectric plate 1021 and a second piezoelectric plate 1022. One face of the first piezoelectric plate 1021, and one face of the second piezoelectric plate 1022 may be fixed inside or outside the air bladder 108 by means of adhesion, preferably all piezoelectric plates are provided on an inner surface of the air bladder 108. All the piezoelectric plates on the apparatus in the present embodiment jointly collect a piezoelectric signal to obtain the above-mentioned Korotkoff sound signals and transmit it to the control module 103.

In this embodiment, multiplexing of the air hose 109 is achieved by placing the connection line in the air hose 109, without the need for separate connection line, it is enough to connect the main unit 200 and the cuff 107 only via the air hose, simplifying a structure of the blood pressure measurement device.

Optionally, the first piezoelectric plate 1021 and the second piezoelectric plate 1022 are provided in a lower half part of the interior of the cuff 107, the first piezoelectric plate 1021 and the second piezoelectric plate 1022 are positioned such that one of them corresponds to the position corresponding to either the left brachial artery or the right brachial artery when blood pressure measurement is performed. A distance between the first piezoelectric plate and the second piezoelectric plate in this embodiment may be 100 mm.

In some optional implementations, the control module 103 includes an analog-to-digital conversion unit and a processing unit.

Specifically, the analog-to-digital conversion unit is used to convert the pressure analog signals to a pressure digital signal, and is used to convert the Korotkoff sound signals to Korotkoff sound digital signals. Illustratively, the analog-to-digital conversion unit may be an ADC interface of an analog-to-digital converter (ADC) chip or an MCU.

The processing unit is used to perform time-frequency transformation processing on the Korotkoff sound digital signals to obtain a first frequency-domain feature, and to perform amplification processing on the first frequency-domain feature to obtain a second frequency-domain feature. The processing unit is further used to shift up the second frequency-domain feature wholly by a preset frequency to obtain a third frequency-domain feature, and is used to perform frequency-time transformation processing on the third frequency-domain feature to obtain a first time-domain feature, and is used to perform amplification processing on the first time-domain feature to obtain Korotkoff sound data.

Specifically, after receiving the Korotkoff sound digital signals, the processing unit performs Fast Fourier Transform (FFT) on the Korotkoff sound digital signals to convert the Korotkoff sound digital signals into a first frequency-domain feature, and then performs amplification processing on the first frequency-domain feature via an operational amplifier, converts the first frequency-domain feature into a second frequency-domain feature, and then shifts up the second frequency-domain feature wholly by a preset frequency, converts the second frequency-domain feature into a third frequency-domain feature, and then, performs Inverse Fast Fourier Transform (IFFT) on the third frequency-domain feature to convert the third frequency-domain feature into a first time-domain feature, and finally performs amplification processing on the first time-domain feature via the operational amplifier, and converts the first time-domain feature into Korotkoff sound data.

Performing amplification processing on the first frequency-domain feature refers to amplification of the first frequency-domain feature by Q times, and Q may be 10, 20, 30, 100 or more than 100, etc. The preset frequency may be determined by a designer according to a response frequency of the audio output module 105. For example, the audio output module 105 is a loudspeaker, and a frequency range of the first frequency-domain feature is 15 Hz to 150 Hz, at this point, the preset frequency may be 30 Hz.

Illustratively, amplification processing may be performed on the first time-domain feature according to the following formula (1).

$$Output\_value = ( 2 \times 32767 \times ( input\_value ) / ( max - min ) - 32767 ) \qquad ( 1 )$$

Where, Output_value denotes Korotkoff sound data, input_value denotes a first time-domain feature, min denotes a minimum value of a sound signal in the first time-domain feature, max denotes a maximum value of the sound signal in the first time-domain feature, and an output numerical range of the sound signal is [−32768, 32767].

Specifically, after receiving the pressure digital signal, the processing unit is further used to process the Korotkoff sound digital signals and the pressure digital signal according to a calibration parameter to obtain blood pressure data.

Illustratively, the blood pressure data may be determined according to the following formula (2).

$$P = ( {P}_{1} - {P}_{0} ) \times K \qquad ( 2 )$$

where, P denotes blood pressure data, P₁ denotes a pressure digital signal, P₀ denotes a calibration parameter, which refers to a pressure digital signal of the air pressure sensor 101 under an ambient atmospheric pressure, K denotes a slope which refers to linearity of the air pressure sensor.

Specifically, after the Korotkoff sound digital signals are determined, the pressure digital signals are selected according to the Korotkoff sound digital signals, and the pressure digital signals corresponding to moments of the Korotkoff sound digital signals are retained, and then the selected pressure digital signals are input into formula (2) to determine the blood pressure data.

In this embodiment, after the Korotkoff sound digital signals are determined, time-frequency transformation, amplification, frequency shift, frequency-time transformation and amplification processing are performed on the Korotkoff sound digital signals, which may improve the problem of poor response of the audio output module 105 due to a fact that a sound frequency of the Korotkoff sounds contains a significant proportion of low-frequency components, and improve a playing effect of the audio output module 105.

Optionally, the control module 103 further includes a notch filter which is used to perform de-noising processing on the Korotkoff sound digital signals to obtain de-noised Korotkoff sound digital signals. At this point, the processing unit is specifically used to perform time-frequency transformation processing on the de-noised Korotkoff sound digital signals to obtain a first frequency-domain feature.

Specifically, after the Korotkoff sound digital signals are determined, a process of converting the Korotkoff sound digital signals to Korotkoff sound data may be as shown in FIG. 8. For the Korotkoff sound digital signals, de-noising processing is first performed via the notch filter, then time-frequency transformation processing is performed via FFT, then amplification processing is performed via a first amplifier (for example, amplifying by 100 times), then frequency shift processing is performed (for example shifting up by 30 Hz), then frequency-time transformation processing is performed via IFFT, and finally amplification processing is performed via a second amplifier (formula (1)) to obtain Korotkoff sound data.

In this embodiment, after the de-noising processing is performed on the Korotkoff sound digital signals, the time-frequency transformation processing is performed on the Korotkoff sound digital signals, which can filter out interference of a 50 Hz power frequency in the Korotkoff sound digital signals, and improve accuracy of the Korotkoff sound data obtained according to the Korotkoff sound digital signals.

Illustratively, after the pressure analog signals and the Korotkoff sound signals are determined, the processing unit is further used to determine a storage time period according to a storage space of the storage module 104, convert the pressure analog signals within the storage time period into pressure digital signals, and obtain air pressure data and blood pressure values by processing the pressure digital signals and the Korotkoff sound signals, and store the air pressure data and the blood pressure in the storage space, and convert the Korotkoff sound signals within the storage time period to Korotkoff sound digital signals, thereby to obtain Korotkoff sound data and store the Korotkoff sound data in the storage space.

In one example, the processing unit is used to determine a duration between a collection moment of a first pressure analog signal and a collection moment of the last pressure analog signal during air bladder depressurization as a storage time period in a case where the storage space of the storage module 104 is greater than a preset storage space. That is, in a case where the storage space of the storage module 104 is greater than the preset storage space, the time period between start and end of collection by the air pressure sensor 101 during air bladder depressurization is determined as a storage time period.

Specifically, the preset storage space is a space required to store all blood pressure data and Korotkoff sound data within a first time period, the first time period being an average duration for completing blood pressure measurement. During blood pressure measurement, the air pressure sensor 101 may collect a plurality of pressure analog signals according to a pressure sampling frequency, and the piezoelectric sensor 102 may collect a plurality of brachial artery pulse signals according to a piezoelectric sampling frequency. If the storage space of the storage module 104 is greater than the preset space, it indicates that the storage space is sufficient. At this point, the processing unit may store all the data collected during air bladder depressurization process in blood pressure testing process in the storage space. The piezoelectric sampling frequency is the same as the pressure sampling frequency.

In another example, the processing unit is used to determine the duration between the Nth second before the start moment of Korotkoff sound data and the Mth second after the disappearance moment of Korotkoff sound data as a storage time period in a case where the storage space of the storage module is less than or equal to the preset storage space, where N≥1, M≥1, N and M may be the same or may be different. That is, the storage time period is consistent with a duration of the above playback data segment, the storage time period is the playback time period described above.

For example, both N and M may be 1 s, 2 s, or 3 s, etc. By taking both N and M being 2 s as an example, the storage time period is a duration between the first 2 s of the first Korotkoff sound and 2 s after the last Korotkoff sound.

In some optional implementations, the control module further includes a control unit, the control unit is used to control display module 106 to synchronously display a pulse icon until the Korotkoff sound disappears or weakens, in a case where the audio output module 105 plays the first Korotkoff sound, or N seconds (N≥1 second) before the first Korotkoff sound appears. At which point a display interface may be as shown in FIG. 9. The heart symbol in the lower half part of FIG. 9 represents the above pulse icon, a number in the lower half part of FIG. 9 represents a pressure at a current moment, a unit for the pressure is millimeter of mercury (mmHg) or kilopascal (Kpa).

Illustratively, the blood pressure measurement device further includes a playback button which is connected to the control module 103 and is used to generate a playback signal according to a preset action, to enable the control module 103 to respond to a playback operation.

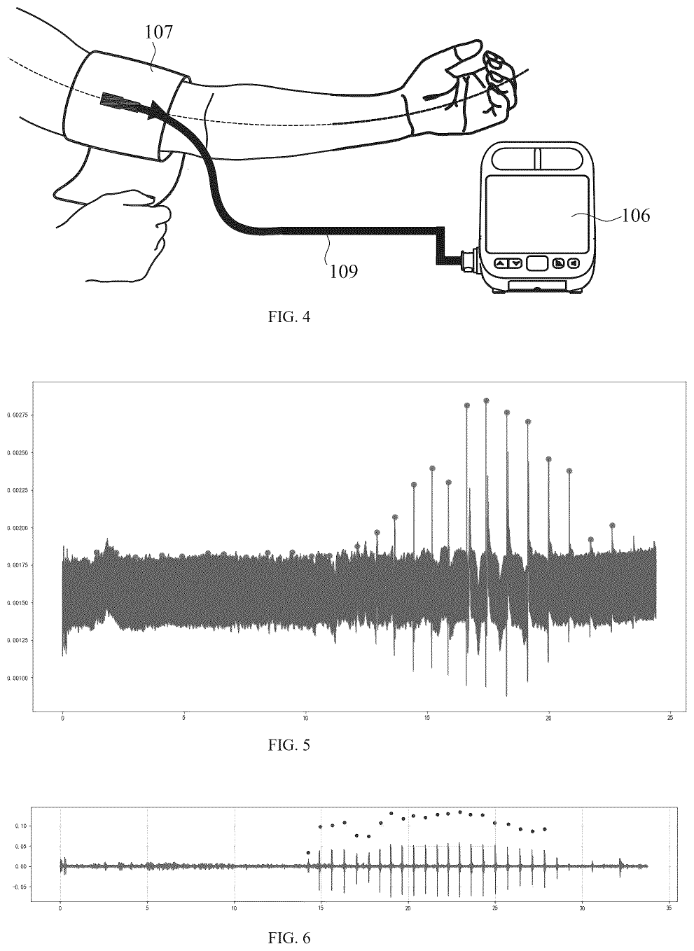

Specifically, the playback button may be set separately, or any of original buttons on the main unit 200 may serve as a playback button. For example, as shown in FIG. 4, any one of the first volume button 201 (increase volume), the second volume button 202 (decrease volume), the control button 203 (start measurement and end measurement), the icon display button 204, and the play button 205 set on the main unit 200 may serve as a playback button.

Preset action that triggers generation of a playback signal may be defined by a designer. For example, the preset action may be performed by pressing long for 3 s, short for 1 s, or pressing three times consecutively, etc. The playback signal may be a low level signal or a high level signal.

Optionally, the blood pressure measurement device further includes a voice assistant, the voice assistant is connected to the control module 103 and generates a playback signal to enable the control module 103 to respond to a playback operation when the voice assistant receives a specific audio (for example “please play back pressure and Korotkoff sounds” or “play data”, etc.).

According to embodiments of the present disclosure, a blood pressure measuring method is provided. It should be noted that the steps illustrated in the flow diagrams of the drawings may be executed in e.g. a computer system includes a set of computer-executable instructions, and, although a logical sequence is shown in a flow diagram, under some circumstances, the shown or described steps may be executed in a sequence different from the sequence here.

A blood pressure measuring method with Korotkoff sounds recognition and playback function is provided in this embodiment, is used for said control module 103, and the method includes:
obtaining pressure analog signals and brachial artery pulse signals in a process in which air pressure within an air bladder drops;
recognizing the brachial artery pulse signals to obtain Korotkoff sound signals;
processing the pressure analog signals to obtain air pressure in the process in which the air pressure inside the air bladder deflates;
processing the pressure analog signals and the Korotkoff sound signals to obtain systolic pressure data and diastolic pressure data; and
in response to a playback operation, synchronously playing back the process in which the air pressure inside the air bladder deflates, a process from the appearance to the disappearance of the Korotkoff sounds, and displaying the systolic pressure data and the diastolic pressure data.

In this embodiment, playing back the process in which the air pressure inside the air bladder deflates is performed by digital display on a display screen; playing back the process from the appearance to the disappearance of the Korotkoff sounds is performed by a sound player or the pulsation of a heartbeat graphic symbol on a display screen, or a combination thereof.

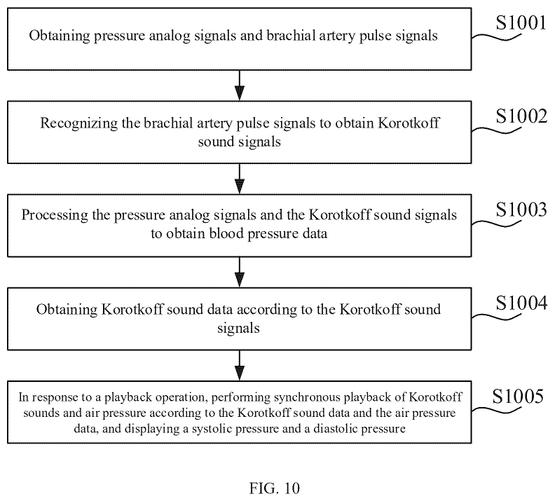

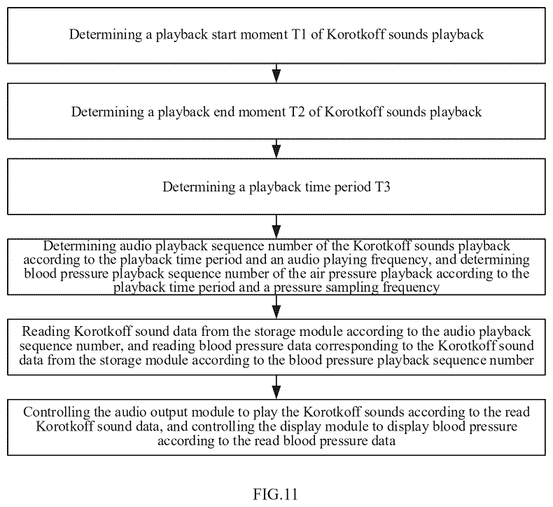

A blood pressure measuring method is provided in another embodiment, and may be used in said control module 103, the control module 103 may be a controller or processor within a device such as a mobile phone, a computer or a computing device, etc. FIG. 10 is a flow schematic diagram of a blood pressure measuring method according to the embodiments of the present disclosure, as shown in FIG. 10, the method includes the following steps:

Step S1001, obtaining pressure analog signals and brachial artery pulse signals.

The pressure analog signals are used to represent air pressure in a cuff, and the brachial artery pulse signals are used to represent sound of blood flowing in a brachial artery during blood pressure measurement process.

Specifically, the control module obtains pressure analog signals from the air pressure sensor, and the control module obtains brachial artery pulse signals from the piezoelectric sensor.

Step S1002, recognizing the brachial artery pulse signals to obtain Korotkoff sound signals.

Specifically, said step S1002 may include the following steps:
Step a1, setting a recognition threshold of the Korotkoff sound signal according to a comparison result between an average arterial signal intensity of the brachial artery pulse signals within a preset time period and a preset threshold.

Specifically, said step a1 includes the following steps:
Step a11, performing band-pass filtering processing on the brachial artery pulse signals within a preset frequency range to obtain arterial intensity signals.

Illustratively, the arterial intensity signals may be as shown in FIG. 5 or FIG. 6. The preset frequency range is an empirical value and may be adjusted according to a demand, for example, a frequency range may be [20 Hz, 120 Hz].
Step a12, obtaining a numerical difference between a maximum value and a minimum value of the arterial intensity signal for each instance in a preset collection time period, and taking the numerical difference as an arterial signal intensity for each instance.

The preset collection time period may be an empirical value and may be adjusted according to an actual need. For example, the preset collection time period may be 1 s or 2 s, etc.
Step a13, obtaining a mean value of the arterial signal intensity cross all instances to obtain an average arterial signal intensity.
Step a14, setting a recognition threshold of Korotkoff sound signals according to a comparison result among the average arterial signal intensity, a first preset threshold and a second preset threshold.

Specifically, the first recognition threshold of the Korotkoff sound signal is set when the average arterial signal intensity is less than the first preset threshold, and the second preset threshold of the Korotkoff sound signal is set to be P times the average arterial signal intensity when the average arterial signal intensity is greater than the second preset threshold. A third recognition threshold of the Korotkoff sound signal is set to be 2P times the average arterial signal intensity when the average arterial signal intensity is between the first preset threshold and the second preset threshold.

P>0, the first preset threshold, the second preset threshold, the first recognition threshold, and P may all be empirical values and may be adjusted according to actual needs. For example, the first preset threshold may be 0.005, the second preset threshold may be 0.008, the first recognition threshold may be 0.015, and P may be 1.5.

Specifically, the average arterial signal intensity between the first preset threshold and the second preset threshold means that the average arterial signal intensity is greater than or equal to the first preset threshold, and the average arterial signal intensity is less than or equal to the second preset threshold.
Step a2, recognizing a Korotkoff sound signal according to the recognition threshold.

Specifically, determining the brachial artery pulse signal, which corresponding to the arterial intensity signal with an arterial signal intensity greater than the recognition threshold, as a Korotkoff sound signal. The recognition threshold may be a first recognition threshold, a second recognition threshold or a third recognition threshold. That is, the control module determines whether the intensity of the arterial intensity signal (that is, arterial signal intensity) for each instance is greater than the recognition threshold. If the arterial signal intensity is greater than the recognition threshold, the arterial intensity signal is determined as a Korotkoff sound signal.
Step S1003, processing the pressure analog signals and the Korotkoff sound signals to obtain blood pressure data, the blood pressure data includes air pressure and blood pressure values of an air bladder pressure deflation process, and the blood pressure values are systolic pressure and diastolic pressure.

Specifically, the control module 103 first selects pressure analog signals according to the Korotkoff sound signals, retains the pressure analog signals corresponding to a collection moment of the Korotkoff sound signals, converts the Korotkoff sound signals and the pressure analog signals corresponding to the Korotkoff sound signals to obtain air pressure data and blood pressure values, so as to ensure that the time to obtain blood pressure data and the time to obtain Korotkoff sound data by subsequent processing are corresponding. That is, the air pressure data are obtained by performing transformation processing on selected pressure analog signals, and a format of the air pressure data is adapted to a format that can be recognized by display module.

Illustratively, the selected pressure analog signals are input to an ADC, and the output of the ADC may be directly determined as blood pressure data, or the blood pressure data may be determined according to the output of the ADC and the above formula (2).
Step S1004, obtaining Korotkoff sound data according to the Korotkoff sound signals.

The Korotkoff sound data is a Korotkoff sound signals after transformation processing, and a format of the Korotkoff sound data is adapted to a format that can be recognized by the audio output module.

Illustratively, the Korotkoff sound signals is input to the ADC, the output of the ADC may be directly determined as Korotkoff sound data, or the output of the ADC after time-frequency transformation, amplification, frequency shift, frequency-time transformation and amplification processing may be determined as Korotkoff sound data.
Step S1005, in response to a playback operation, performing synchronous playback of Korotkoff sounds and air pressure according to the Korotkoff sound data and the air pressure data, and displaying the systolic pressure and the diastolic pressure.

In the blood pressure measuring method provided in this embodiment, the Korotkoff sounds are recognized to obtain blood pressure values, and the Korotkoff sounds are played back to judge whether the blood pressure values are accurate. After obtaining a pressure analog signals and brachial artery pulse signals, a Korotkoff sound signals are recognized from the brachial artery pulse signals, and then blood pressure data is determined according to the Korotkoff sound signals and the pressure analog signals, Korotkoff sound data is determined according to the Korotkoff sound signals, pressure data is obtained according to the pressure analog signals, and corresponding air pressure data when the Korotkoff sounds appears and corresponding air pressure data when the Korotkoff sounds disappears in the blood pressure data are serve as a blood pressure measurement result, the accuracy of the determined Korotkoff sound data and the air pressure data corresponding to the Korotkoff sound data can be improved, more accurate original measurement data for determining blood pressure values is provided via a playback operation, thereby to improve accuracy of blood pressure measurement, Korotkoff sound data consistent with a manual auscultatory method is provided for a user to verify blood pressure values by playing back the Korotkoff sounds, and original data is provided for verifying blood pressure values, analyzing a user's blood pressure measurement process, and thus for disease analysis.

Specifically, when a user performs a playback operation (such as pressing a playback button with a preset action), the control module, in response to the playback operation, controls the audio output module to perform Korotkoff sounds playback of the Korotkoff sound data read from the storage module, and controls the display module to perform air pressure synchronous playback of the air pressure data and display blood pressure values at the same time, in which the blood pressure data is corresponding to the Korotkoff sound data and read from the storage module. The Korotkoff sounds played by the audio output module corresponds to the air pressure displayed by the display module.

In this embodiment, after blood pressure measurement is performed, the Korotkoff sounds in the measurement process may further be played back at any time, air pressure values corresponding to the Korotkoff sounds during measurement is displayed, and blood pressure values are displayed, which is convenient for medical staff to view and analyze collected data many times, thereby to determine a user's blood pressure more accurately, which helps medical staff to judge more accurately whether there is a phenomenon such as arrhythmia or atrial fibrillation.

Illustratively, in order to ensure that the played Korotkoff sounds corresponds to a displayed air pressure, the blood pressure measuring method further includes steps c1 to c5 after a user triggers the playback operation:
Step c1, determining a playback data segment and a playback time period according to the pressure analog signals and Korotkoff sound signals.

The playback data segment includes a data segment of Korotkoff sounds playback and a data segment of blood pressure playback, and the playback time period is a time period in which the Korotkoff sound data and the blood pressure data are played back simultaneously.

Specifically, playback time period T3 is a difference between a playback end moment T2 of Korotkoff sounds playback and a playback start moment T1 of Korotkoff sounds playback, that is, T3=T2−T1, the playback end moment T2 of Korotkoff sounds playback and the playback start moment T1 of Korotkoff sounds playback are determined according to playback data segments.

When the data segment of the Korotkoff sounds playback includes Korotkoff sound data from the Nth second before a start moment of the Korotkoff sound data to the Mth second after a disappearance moment of the Korotkoff sound data, the playback end moment is the Mth second after the disappearance moment of the Korotkoff sound data, and the playback start moment is the Nth second before the starting moment of the Korotkoff sound data.
Step c2, determining audio playback sequence number of the Korotkoff sounds playback according to the playback time period and an audio playback frequency.

Specifically, the audio playback sequence number may be the product of the playback time period T3 and the audio playing frequency f2, i.e., the audio playback sequence number is (T3×f2).
Step c3, determining air pressure playback sequence number of the air pressure playback according to the playback time period and a pressure sampling frequency.

Specifically, the air pressure playback sequence number may be the product of the playback time period T3 and the pressure sampling frequency f1, i.e., the air pressure playback sequence number is (T3×f1).

The audio playback sequence number and the air pressure playback sequence number is used to establish a corresponding relationship between Korotkoff sound data and blood pressure data at the same moment in the playback time period.
Step c4, reading the Korotkoff sound data from the storage module according to the audio playback sequence number.
Step c5, reading the blood pressure data corresponding to the Korotkoff sound data from the storage module according to the air pressure playback sequence number.

Specifically, audio playback sequence number and air pressure playback sequence number corresponding to a specific playing moment are determined according to the playback time period, then data is read from the storage module according to the audio playback sequence number and the air pressure playback sequence number, which can ensure that the read Korotkoff sound data corresponds to the pressure data.

Illustratively, after receiving a playback signals, the process in which the control module plays Korotkoff sounds and displays a blood pressure may be as shown in FIG. 11, firstly, the control module determines the playback start time T1 of the Korotkoff sounds playback and the playback end time T2 of the Korotkoff sounds playback, and then the playback time period T3 may be determined according to T1 and T2.

After that, audio playback sequence number of the Korotkoff sounds playback are determined according to the playback time period and an audio playback frequency, and air pressure playback sequence number of the air pressure playback are determined according to the playback time period and a pressure sampling frequency, then Korotkoff sound data is read from the storage module according to the audio playback sequence number, and the air pressure data corresponding to the Korotkoff sound data is read from the storage module according to the air pressure playback sequence number, after that, the audio output module is controlled to play the Korotkoff sounds according to the read Korotkoff sound data, and the display module is controlled to display air pressure according to the read air pressure data, and to display the corresponding air pressure recognized as a systolic pressure when the Korotkoff sound appears, and to display the corresponding air pressure recognized as a diastolic pressure when the Korotkoff sounds disappears. In which, the audio playback sequence number and the air pressure playback sequence number is used to establish a corresponding relationship between Korotkoff sound data and blood pressure data at the same moment in the playback time period.

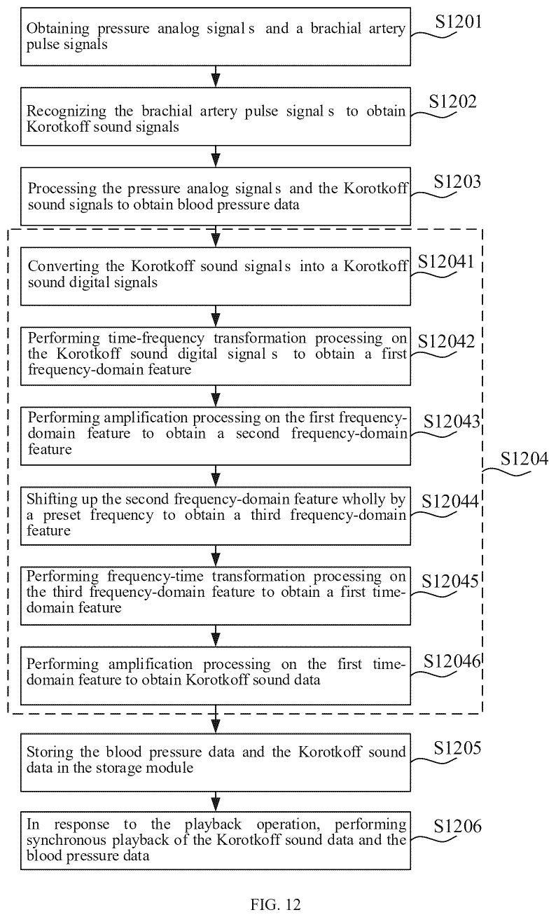

A blood pressure measuring method is provided in this embodiment, and may be used for the control module 103. FIG. 12 is a flow schematic diagram of another blood pressure measuring method according to the embodiments of the present disclosure. As shown in FIG. 12, the method includes the following steps:
Step S1201, obtaining pressure analog signals and brachial artery pulse signals.

For details, please refer to Step S1001 in the embodiment shown in FIG. 10, details are not repeated here.
Step S1202, recognizing the brachial artery pulse signals to obtain Korotkoff sound signals.

For details, please refer to Step S1002 in the embodiment shown in FIG. 10, details are not repeated here.
Step S1203, processing the pressure analog signals and the Korotkoff sound signals to obtain blood pressure data, the blood pressure data includes air pressure and blood pressure values of an air bladder pressure deflation process.

For details, please refer to Step S1003 in the embodiment shown in FIG. 10, details are not repeated here.
Step S1204, obtaining Korotkoff sound data according to the Korotkoff sound signals.

Specifically, the step S1204 includes:
Step S12041, converting the Korotkoff sound signals into Korotkoff sound digital signals.

Specifically, the brachial artery pulse signals may be converted into a Korotkoff sound digital signals via an ADC chip or an ADC interface of a MCU.

It should be understood that the pressure analog signals may also be converted into a pressure digital signal via the ADC chip or the ADC interface of the MCU.
Step S12042, performing time-frequency transformation processing on the Korotkoff sound digital signals to obtain a first frequency-domain feature.

Specifically, FFT is performed on the Korotkoff sound digital signals to convert the Korotkoff sound digital signals into a first frequency-domain feature.
Step S12043, performing amplification processing on the first frequency-domain feature to obtain a second frequency-domain feature.

Specifically, amplification processing may be performed on the first frequency-domain feature via an operational amplifier to obtain a second frequency-domain feature. Amplification factor is greater than or equal to 10, for example, amplification factor may be 10, 20, 50, 80 or 100, etc.
Step S12044, shifting up the second frequency-domain feature wholly by a preset frequency to obtain a third frequency-domain feature.
Step S12045, performing frequency-time transformation processing on the third frequency-domain feature to obtain a first time-domain feature.

Specifically, IFFT is performed on the third frequency-domain feature to convert the third frequency-domain feature into a first time-domain feature.
Step S12046, performing amplification processing on the first time-domain feature to obtain Korotkoff sound data.

Specifically, amplification processing may be performed on the first time-domain feature via the above formula (1), thereby to obtain Korotkoff sound data.

Further, in some optional implementations, after step S12041 and before step S12042, the blood pressure measuring method further includes: performing de-noising processing on the Korotkoff sound digital signals to obtain de-noised Korotkoff sound digital signals. At this point, the step S12042 specifically is: performing time-frequency transformation processing on the de-noised Korotkoff sound digital signals to obtain a first frequency-domain feature.

Specifically, de-noising processing may be performed on the Korotkoff sound digital signals via a notch filter, which filters out interference of a 50 Hz power frequency in the Korotkoff sounds digital signals, and improves accuracy of the Korotkoff sound data obtained according to according to the Korotkoff sounds digital signals.
Step S1205, storing the blood pressure data and the Korotkoff sound data in the storage module.
Step S1206, in response to the playback operation, performing synchronous playback of the Korotkoff sound data and the blood pressure data, the blood pressure data including air pressure of an air bladder pressure deflation process and blood pressure values.

For details, please refer to the step b2 in the above embodiment, details are not repeated here.

In some optional implementations, after the Korotkoff sound data and the air pressure data are determined and before the playback operation is performed, the blood pressure measuring method further includes: determining a blood pressure measurement result according to the air pressure data and the Korotkoff sound data, and controlling the control display module to display the blood pressure measurement result.

The blood pressure measurement result includes a low pressure signal and a high pressure signal, to achieve measurement of a high blood pressure and a low blood pressure. The high pressure signal refers to a blood pressure value corresponding to the first Korotkoff sound during blood pressure measurement, i.e., a systolic pressure, and the low pressure signal refers to a blood pressure value corresponding to disappearance or change of Korotkoff sounds during blood pressure measurement, i.e., a diastolic pressure. Therefore, the blood pressure data in this embodiment includes the blood pressure values as a blood pressure measurement result, and the air pressure during air bladder depressurization, and synchronously playing back the Korotkoff sounds and the blood pressure data includes synchronously playing back the Korotkoff sounds, the air pressure during air bladder depressurization, and displaying the blood pressure values at the same time.

In the blood pressure measuring method provided by this embodiment, after the Korotkoff sound signals are converted into a Korotkoff sound digital signals, time-frequency transformation, amplification, frequency shift, frequency-time transformation and amplification processing are performed on the Korotkoff sound digital signals, which may improve the problem of poor response of the audio output module due to a fact that a sound frequency of the Korotkoff sounds contains a significant proportion of low-frequency components, and improve a playing effect of the audio output module.

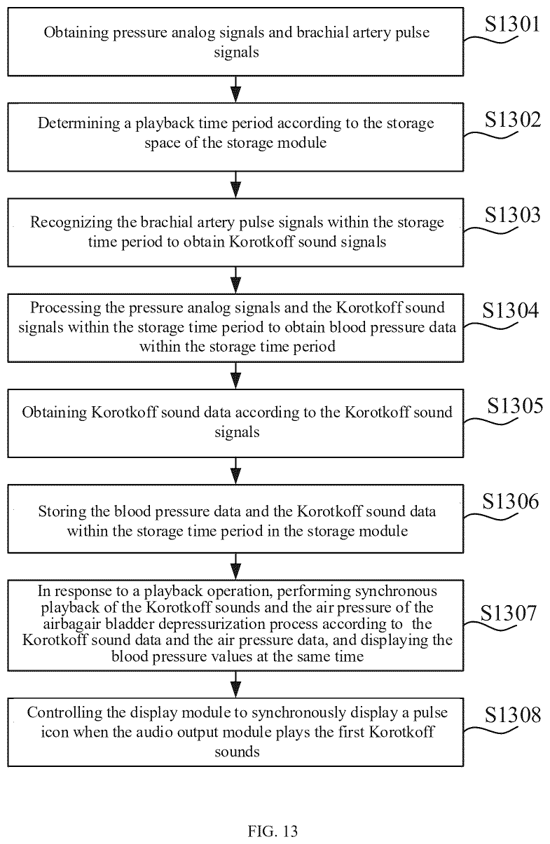

A blood pressure measuring method is provided in this embodiment, and may be used for the control module 103. FIG. 13 is a flow schematic diagram of another blood pressure measuring method according to the embodiments of the present disclosure, as shown in FIG. 13, the method includes the following steps:
Step S1301, obtaining pressure analog signals and brachial artery pulse signals.

For details, please refer to Step S1001 in the embodiment shown in FIG. 10, details are not repeated here.
Step 1302, determining a storage time period according to the storage space of the storage module.

In some implementations, a duration between a collection moment of a first pressure analog signal and a collection moment of the last pressure analog signal during air bladder depressurization process is determined as a storage time period in a case where the storage space of the storage module is greater than a preset storage space. That is, in a case where the storage space of the storage module is greater than the preset storage space, the time period between start and end of collection by the air pressure sensor 101 is determined as a storage time period.

Specifically, the preset storage space is a space required to store all blood pressure data and Korotkoff sound data within a first time period, and the first time period being an average duration for completing blood pressure measurement.

In this implementation, all measurement data may be stored to ensure data integrity and avoid data omissions.

In some other implementations, the duration between the Nth second before the start moment of Korotkoff sound data and the Mth second after the disappearance moment of Korotkoff sound data is determined as a storage time period, in a case where the storage space of the storage module is less than or equal to the preset storage space, where N≥1, M≥1, N and M may be the same or may be different. That is, the storage time period is consistent with a duration of the above playback data segment, the storage time period is the playback time period described above.

For example, both N and M may be 1 s, 2 s, or 3 s, etc. By taking both N and M being 2 s as an example, the storage time period is a duration between the first 2 s of the first Korotkoff sound and 2 s after the last Korotkoff sound.

In this embodiment, only the measurement data between the first and second moments may be stored, reducing the amount of data and saving a storage space.
Step S1303, recognizing the brachial artery pulse signals within the storage time period to obtain Korotkoff sound signals.

For details, please refer to step S1002 in the embodiment shown in FIG. 10, details are not repeated here.
Step S1304, processing the pressure analog signals and the Korotkoff sound signals within the storage time period, to obtain blood pressure data within the storage time period.

For details, please refer to step S1003 in the embodiment shown in FIG. 10, details are not repeated here.
Step S1305, obtaining Korotkoff sound data according to the Korotkoff sound signals.

For details, please refer to step S1204 in the embodiment shown in FIG. 12, details are not repeated here.
Step S1306, storing the blood pressure data and the Korotkoff sound data within the storage time period in the storage module.
Step S1307, in response to a playback operation, performing synchronous playback of the Korotkoff sounds and the air pressure of the air bladder depressurization process according to the Korotkoff sound data and the air pressure data, and displaying the blood pressure values at the same time.

For details, please refer to the step b2 in the above embodiment, details are not repeated here.
Step S1308, controlling the display module to synchronously display a pulse icon when the audio output module plays the first Korotkoff sound.

Illustratively, the display interface including a pulse icon may be as shown in FIG. 9.

In the blood pressure measuring method provided in this embodiment, a storage time period is determined before processing the pressure analog signals and the brachial artery pulse signals, which can flexibly adjust a length of stored data and avoid a situation of not being stored. The display module is controlled to synchronously display a pulse icon in a case where the audio output module plays the first Korotkoff sound, or at S seconds before the first Korotkoff sound appears, in which S≥1 second, thereby enabling medical staff to more accurately determine the user's actual blood pressure.

A control module is further provided in this embodiment, the control module being used to implement the above embodiments and the optional implementations, contents which have been already described are not repeated. As used below, the term “module” may be a combination of software and/or hardware that implements a predetermined function. Although the apparatus described in the following embodiment is better implemented in software, implementation of hardware or a combination of software and hardware may also be possible and conceived.

The control module in this embodiment is presented in the form of a functional unit. The unit here refers to an Application Specific Integrated Circuit (ASIC), a processor that executes one or more software or fixed programs, and a memory, and/or other devices that can provide the above function.

Embodiments of the present disclosure further provide an electronic device, wherein the main unit 200 mentioned above may be an electronic device.

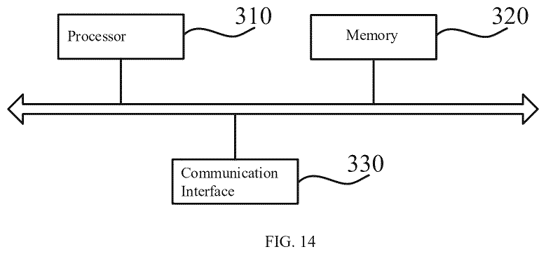

As shown in FIG. 14, the electronic device includes one or more processors 310, a memory 320, and a communication interface 330 for connecting components, including a high-speed interface and a low-speed interface. The components are communicatively connected to each other using different buses, and may be installed on a common mainboard or installed by other means as required. A processor may process instructions executed within the electronic device, including instructions stored in or on the memory to display graphical information of a GUI on an external input/output device (such as a display device coupled to an interface). In some optional implementations, if needed, a plurality of processors and/or multiple buses may be used together with a plurality of memories. Similarly, a plurality of electronic devices may be connected, each of the electronic devices providing some of necessary operations (for example, as an array of servers, a group of blade servers, or a multiprocessor system). In FIG. 14, a processor 310 is taken as an example. The processor 310 is equivalent to the above control module 103, and the memory 320 is equivalent to the above storage module 104.

In the description of the present Specification, reference terms “this embodiment”, “an embodiment”, “some embodiments”, “an example”, “a specific example” or “some examples”, etc. are used to mean that a specific feature, structure, material or characteristic described in conjunction with the embodiment or example is contained in at least one embodiment or example of the present disclosure. In the present Specification, schematic representations of the above terms are not necessary to aim at the same embodiments or examples. And, the described specific feature, structure, material or characteristic may be combined in a suitable manner in any one or more embodiments or examples. In addition, without contradicting each other, persons skilled in the art may incorporate and combine different embodiments or examples described in the present Specification as well as the features of different embodiments or examples.

Although the embodiments of the present disclosure are described with reference to the drawings, persons skilled in the art may make various modifications and variants without deviating from the spirit and scope of the present disclosure, and such modifications and variants fall within the scope defined by the present disclosure.

What is claimed is:

1. A blood pressure measurement device with Korotkoff sounds recognition and playback function, wherein the blood pressure measurement device comprises a control module, an air pressure sensor and a vibration sensor;
the air pressure sensor being configured to collect a pressure analog signals which is used to characterize air pressure inside an air bladder;
the vibration sensor being configured to be fixed in a cuff and used to collect a brachial artery pulse signals; and
the control module being configured to control the air pressure inside the air bladder and recognize Korotkoff sounds in the brachial artery pulse signals collected by the vibration sensor, and obtain systolic pressure data and diastolic pressure data according to the Korotkoff sounds and the air pressure inside the air bladder; and the control module being further configured to play back and display, in response to a playback operation, air pressure deflation process inside the air bladder, a process from the appearance to the disappearance of the Korotkoff sounds, and the systolic pressure data and the diastolic pressure data.

2. The blood pressure measurement device according to claim 1, wherein the vibration sensor is a piezoelectric sensor or a microphone.

3. The blood pressure measurement device according to claim 1, wherein playback of the air pressure deflation process inside the air bladder is displayed digitally, and playback of the process from the appearance to the disappearance of the Korotkoff sounds is performed via a sound player and/or via pulsation of a heartbeat graphic symbol on a display screen.

4. The blood pressure measurement device according to claim 1, wherein the control module is further used to transmit the Korotkoff sounds, the air pressure inside the air bladder, the systolic pressure data and the diastolic pressure data, and a corresponding measurement time as associated blood pressure measurement data to an external device for storage and playback.

5. The blood pressure measurement device according to claim 4, wherein the playback operation comprises selecting blood pressure measurement data needing to be played back to perform a playback operation.

6. A blood pressure measurement device comprising an air pressure sensor, a piezoelectric sensor and a control module, wherein:
the air pressure sensor is configured to collect pressure analog signals which is configured to represent air pressure inside an air bladder;
the piezoelectric sensor is configured to be fixed in a cuff and configured to collect brachial artery pulse signals; and
the control module is configured to obtain the pressure analog signals and the brachial artery pulse signals, recognize the brachial artery pulse signals to obtain Korotkoff sound signals, and obtain Korotkoff sound data according to the Korotkoff sound signals, and the control module being further configured to process the pressure analog signals and the Korotkoff sound signals to obtain blood pressure data which includes air pressure and blood pressure values of a pressure deflation process in the air bladder, the blood pressure values being systolic pressure and diastolic pressure,
the control module being further configured to respond to a playback operation to perform synchronous playback of the Korotkoff sounds and the air pressure, and display the systolic pressure and the diastolic pressure,
wherein the air pressure sensor and the piezoelectric sensor are respectively connected with the control module for communication.

7. The blood pressure measurement device according to claim 6, wherein the blood pressure measurement device further comprises a storage module, an audio output module and a display module; and
the control module is further configured to store the Korotkoff sound data and the blood pressure data into the storage module to control the audio output module to perform Korotkoff sounds playback of the Korotkoff sound data read from the storage module, and control the display module to perform air pressure playback and blood pressure values display of blood pressure data corresponding to the Korotkoff sound data read from the storage module; wherein the storage module, the audio output module and the display module are respectively connected with the control module for communication.

8. The blood pressure measurement device according to claim 7, wherein the control module is configured to determine a playback data segment and a playback time period according to the pressure analog signals and the Korotkoff sound signal; and
wherein the playback data segment comprises a data segment of the Korotkoff sounds playback and a data segment of the air pressure playback, and the playback time period is a time period during which the Korotkoff sound data and the blood pressure data are synchronously played back.

9. The blood pressure measurement device according to claim 7, wherein the control module is configured to determine audio playback sequence number of the Korotkoff sounds playback according to the playback time period and an audio playback frequency of the Korotkoff sounds, and determine air pressure playback sequence number of the air pressure playback according to the playback time period and a pressure sampling frequency of the air pressure.

10. The blood pressure measurement device according to claim 8, wherein the data segment of the Korotkoff sounds playback comprises Korotkoff sound data from the Nth second before a start moment of the Korotkoff sound data to the Mth second after a disappearance moment of the Korotkoff sound data; and the data segment of the blood pressure playback is blood pressure data corresponding to a data segment of the Korotkoff sounds playback, the playback time period is consistent with a duration of the playback data segment; where N≥1, and M≥1.

11. The blood pressure measurement device according to claim 8, wherein the playback time period is a difference between a playback end moment of the Korotkoff sounds playback and a playback start moment of the Korotkoff sounds playback, the playback end moment is the Mth second after a disappearance moment of the Korotkoff sound data, and the playback start moment is the Nth second before a start moment of the Korotkoff sound data, where N≥1 and M≥1.

12. The blood pressure measurement device according to claim 6, wherein the control module is further configured to set a recognition threshold of the Korotkoff sound signals according to a comparison result between an average arterial signal intensity of the brachial artery pulse signals within a preset time period and a preset threshold, and recognize Korotkoff sound signals according to the recognition threshold.

13. The blood pressure measurement device according to claim 12, wherein the control module is further configured to perform band-pass filtering processing on the brachial artery pulse signals within a preset frequency range to obtain an arterial intensity signal, and the control module is configured to obtain a numerical difference between a maximum value and a minimum value of the arterial intensity signals within a preset collection time period for each instance, and take the numerical difference as an arterial signal intensity for each instance; the control module is further configured to obtain a mean value of the arterial signal intensity across all instances to obtain an average arterial signal intensity, and is configured to set a recognition threshold of Korotkoff sound signals according to a comparison result among the average arterial signal intensity, a first preset threshold and a second preset threshold.

14. The blood pressure measurement device according to claim 13, wherein the control module is configured to set the recognition threshold of the Korotkoff sound signals to be a first recognition threshold when the average arterial signal intensity is less than the first preset threshold, and the control module is configured to set the second preset threshold of the Korotkoff sound signals to be P times the average arterial signal intensity when the average arterial signal intensity is greater than the second preset threshold, and the control module is further configured to set a third recognition threshold of the Korotkoff sound signals to be 2P times the average arterial signal intensity when the average arterial signal intensity is between the first preset threshold and the second preset threshold, where P>0.

15. The blood pressure measurement device according to claim 13, wherein the control module is configured to determine the arterial intensity signal with the arterial signal intensity being greater than the recognition threshold as a Korotkoff sound signal.

16. A blood pressure measuring method, wherein the method comprises:
obtaining pressure analog signals and brachial artery pulse signals;
recognizing the brachial artery pulse signals to obtain Korotkoff sound signals, and obtaining Korotkoff sound data according to the Korotkoff sound signals;
processing the pressure analog signals and the Korotkoff sound signals to obtain blood pressure data which comprises air pressure of an air bladder pressure deflation process and blood pressure values, wherein the blood pressure values are systolic pressure and diastolic pressure; and
in response to a playback operation, performing synchronous playback of Korotkoff sounds and air pressure according to the Korotkoff sound data and the air pressure data, and displaying the systolic pressure and the diastolic pressure.

17. The method according to claim 16, wherein recognizing the brachial artery pulse signals to obtain Korotkoff sound signals comprises:
setting a recognition threshold of the Korotkoff sound signals according to a comparison result between an average arterial signal intensity of the brachial artery pulse signals within a preset time period and a preset threshold; and
recognizing Korotkoff sound signals according to the recognition threshold.

18. The method according to claim 17, wherein setting a recognition threshold of the Korotkoff sound signal according to a comparison result between an average arterial signal intensity of the brachial artery pulse signals within a preset time period and a preset threshold comprises:
performing band-pass filtering processing on the brachial artery pulse signals within a preset frequency range to obtain an arterial intensity signal;
obtaining a numerical difference between a maximum value and a minimum value of the arterial intensity signal within a preset collection time period for each instance, and taking the numerical difference as an arterial signal intensity for each instance;
obtaining a mean value of the arterial signal intensity across all instances to obtain an average arterial signal intensity; and
setting a recognition threshold of a Korotkoff sound signal according to a comparison result among the average arterial signal intensity, a first preset threshold and a second preset threshold.
